(19) United States
(12) Patent Application Publication
(10) Pub. No.: US 2008/0022754 A1
(43) Pub. Date: Jan. 31, 2008

(54) Gas sensor with increased sealing performance

(75) Inventor: Kazuya Nakagawa, Kariya-shi (JP)

Correspondence Address: NIXON & VANDERHYE, PC, 901 NORTH GLEBE ROAD, 11TH FLOOR, ARLINGTON, VA, 22203, US

(73) Assignee: Denso Corporation, Kariya-city (JP)

(21) Appl. No.: 11/802,970

(22) Filed: May 29, 2007

(30) Foreign Application Priority Data
Jul. 31, 2006 (JP) 2006-209023

(51) Int. Cl. G01N 33/00

(52) U.S. Cl. 73/31.05

(57) ABSTRACT

A gas sensor is disclosed as having a gas sensing element operative to detect a concentration of a specified gas in measuring gases, a cylindrical housing internally supporting the gas sensing element in fixed place, and a cylindrical measuring gas side cover fixedly secured to the housing at a leading end thereof so as to cover a leading end of the gas sensing element. The gas sensor has a response, ranging from 150 ms to 200 ms, which is a parameter representing a speed of detecting the concentration of the specified gas with respect to variation in a specified gas concentration in the measuring gases. The measuring gas side cover has fine holes each with an opening surface area ranging from 0.1 mm² to 1 mm².

### CROSS REFERENCE TO RELATED APPLICATIONS

This application is related to Japanese Patent Application No. 2006-209023, filed on Jul. 31, 2006, the content of which is hereby incorporated by reference.

### BACKGROUND OF THE INVENTION

1. Technical Field of the Invention

The present invention relates to gas sensors and, more particularly, to a gas sensor mounted on an exhaust pipe or the like of an internal combustion to be exposed to measuring gases for measuring a concentration of a specified gas.

2. Description of the Related Art

Gas sensors have heretofore been known as sensors to be mounted on exhaust pipes of internal combustion engines of motor vehicles and utilized for controlling an air/fuel ratio of an air fuel mixture in the engine. One example of such gas sensors is disclosed in Japanese Patent Application Publication No. 5-149914 related to a gas sensor of the type in which atmospheric air is introduced.

With the gas sensor of such type mounted on the exhaust pipe of the internal combustion engine, a leading end of the gas sensor is exposed to measuring gases. Further, the gas sensor has a cover for protecting a gas sensing element operative to detect a concentration of specified gas in measuring gases. Moreover, the cover is formed with a number of gas ventilation holes to pass measuring gases therethrough to the gas sensing element for detecting variation in measuring gases with high voltage. However, during passage of measuring gases through the gas ventilation holes, water droplets prevailing in the exhaust pipe penetrate through the gas ventilation holes of the cover to an inside area of the cover. Thus, the gas sensing element, elevated at high temperatures, suffer the water droplets. This causes a damage to occur on the gas sensing element with a resultant degradation in response of the gas sensing element.

Meanwhile, for addressing the tasks of a water-incursion resistance and response of the gas sensing element, it is effective to allow the cover to be formed with a large number of small gas ventilation holes. However, the provision of such a large number of ventilation holes results in the occurrence of an issue with a drop in strength of the cover. Thus, the cover encounters a difficulty in providing a large number of small ventilation holes.

### SUMMARY OF THE INVENTION

The present invention has been completed with a view to addressing the above issues and has an object to provide a gas sensor that has increased water-incursion resistance for thereby effectively preventing a gas sensing element from being damaged.

To achieve the above object, a first aspect of the present invention provides a gas sensor comprising a gas sensing element operative to detect a concentration of a specified gas in measuring gases, a cylindrical housing internally supporting the gas sensing element in fixed place, and a cylindrical measuring gas side cover fixedly secured to the housing at a leading end thereof so as to cover a leading end of the gas sensing element. The gas sensor has a response, ranging from 150 ms to 200 ms, which is a parameter representing a speed of detecting the concentration of the specified gas with respect to variation in a specified gas concentration in the measuring gases. The measuring gas side cover has fine holes each with an opening surface area ranging from 0.1 mm² to 1 mm².

According to the present invention, the gas sensor has a response, ranging from 150 ms to 200 ms, which is a parameter representing a speed of detecting the concentration of the specified gas with respect to variation in a specified gas concentration in the measuring gases. Further, the measuring gas side cover has fine holes each with an opening surface area ranging from 0.1 mm² to 1 mm². These are parameters obtained upon experimental tests conducted by the inventor of the present patent application. The experimental tests have been conducted in a sequence described below. That is, first, the gas sensor implementing the present invention was mounted on an exhaust pipe of a motor vehicle. Then, the engine was started up into a steady state with exhaust gases maintained at a fixed temperature, under which exhaust gases are altered in composition from a rich state to a lean state. Then, measurement is made on a time period in which upon altering the composition of exhaust gases, a variation takes place in an output of the gas sensor from a rich side to a lean side.

With the gas sensor having a response less than 150 ms, the response of the gas sensor is impractical with a resultant difficulty of accurately detecting a concentration of the specified gas in measuring gases. With the gas sensor having a response exceeding 200 ms, further, the response of the gas sensor is adequate in practical use but has less water-incursion resistance in practical use. Therefore, a moisture penetrates through fine holes, formed in a measuring gas side cover, into an inside of the cover to adhere onto the leading end of the gas sensing element. This causes cracking to occur on the gas sensing element, resulting in a difficulty of precisely detecting the concentration of specified gas.

If the fine hole, formed in the measuring gas side cover, has an opening surface area less than 0.1 mm², the gas sensor has favorable water-incursion resistance. This makes it possible to effectively precluding moisture, penetrated to the inside of the cover through the fine hole formed in the measuring gas side cover, from adhering onto the gas sensing element. However, the response of the gas sensor is impractical in use with a resultant difficulty of precisely detecting the concentration of specified gas. In addition, if the fine hole, formed in the measuring gas side cover, has an opening surface area greater than 1 mm², the gas sensor has a favorable response but water-incursion resistance of the gas sensor is impractical. This causes moisture, penetrated to the inside of the cover through the fine hole formed in the measuring gas side cover, to adhere onto the gas sensing element. This results in a fear of cracking occurring on the gas sensing element with a resultant difficulty of precisely detecting the concentration of specified gas.

FIG. 11 is a graph representing the relationship between an opening surface area of each of and the number of fine holes, formed in a measuring gas side cover, and a response and water-incursion resistance. As shown in FIG. 11, in order to obtain a response in the order of 150 ms that is practical in use, the measuring gas side cover needs to have the fine holes in the number of pieces greater than 600 in case of the cover having the fine holes each with 0.1 mm² and have the fine holes in the number of pieces greater than 60 in case of the cover having the fine holes each with 1 mm² while the number of the fine holes needs to be greater 6 in case of the cover having the fine holes each with 10 mm². However, with the fine holes each with 1 mm², water-incursion resistance of the gas sensor is impractical in use.

With the present invention, accordingly, the gas sensor is arranged to include a measuring gas side cover configured to provide a response ranging from 150 ms to 200 ms while having fine holes each with an opening surface area ranging from 0.1 mm² to 1 mm². This enables the gas sensor to have advantages with both of a response and water-incursion resistance that are practical in use.

With the gas sensor of the present embodiment, the measuring gas side cover may be preferably made of a mesh-like member composed of wire components woven with a clearance equal to or less than 1 mm, and the wire components may be made of stainless steel wires each having a diameter equal to or greater than 0.3φ.

With such a structure, the mesh-like member is composed of the wire components with the clearance equal to or less than 1 mm. Thus, the gas sensor of the present embodiment has increased water-incursion resistance. This effectively precludes water droplets from penetrating from the outside into the inside area of the measuring gas side cover, enabling the gas sensor to have increased operating life while having increased reliability in operation.

Further, it becomes possible to provide a gas sensor that can prevent a gas sensing element from suffering water even when used under high temperature environments.

Moreover, the measuring gas side cover may be preferably and suitably formed in any one of optimum shapes.

For instance, the measuring gas side cover may preferably have a saclike configuration. In forming the measuring gas side cover of such a configuration, wire components are woven into a mesh-like sheet, which in turn is pressed against a dome-shaped die, making it easy to fabricate the cover into the saclike configuration with the sheet being maintained in a uniform mesh pattern.

Further, the measuring gas side cover may preferably have a cone-shaped configuration. In forming the measuring gas side cover of such a configuration, the wire components are woven into the mesh-like sheet, which in turn is wound on a cone-shaped die, making it easy to fabricate the cover into the cone-shaped configuration.

Furthermore, the measuring gas side cover may preferably have a cylindrical configuration with a leading end thereof being shackled and closed. In forming the measuring gas side cover of such a configuration, the wire components are woven into the mesh-like sheet, which in turn is processed in a cylindrical shape and a leading end thereof is shackled and closed in a final shape in easy fabrication.

Moreover, the measuring gas side cover may be preferably formed in a cylindrical shape and includes a cylindrical metallic plate body and a mesh-like cylindrical body, composed of woven wire components, which is connected to one end of the metallic plate body.

In addition, the measuring gas side cover may be preferably formed in a cylindrical shape and include an inner cover formed in a cylindrical shape and disposed in an inside area, wherein the inner cover has a gas ventilation bore providing fluid communication between inside and outside areas, and wherein the metallic plate body acts as an outer cover that is radially spaced from the inner cover so as to cover the gas ventilation bore. With the measuring gas side cover of such a structure, the gas ventilation bore formed in the inner cover can be protected with the cylindrical metallic plate body of the outer cover, enabling the gas sensor to have increased water-incursion resistance.

Further, the measuring gas side cover may preferably include a multi-layer structure formed in a cylindrical shape having two kinds of an inner cover and an outer cover, wherein the inner cover includes a mesh-like member formed by weaving wire components, and wherein the outer cover is made of a metallic plate and has a gas ventilation bore.

With the gas sensor having the measuring gas side cover of such a structure, measuring gases enter the inside of the cover through the gas ventilation bore formed in the outer cover. Measuring gases then pass through the clearances among the wire components formed in the inner cover on an entire area thereof to reach the gas sensing element, causing the gas sensor to have increased response.

Furthermore, the measuring gas side cover may preferably include a multi-layer structure formed in a cylindrical shape having two kinds of an inner cover and an outer cover, wherein the inner cover is made of a metallic plate and has a gas ventilation bore, and wherein the outer cover includes a mesh-like member formed by weaving wire components.

With the gas sensor having the measuring gas side cover of such a structure, the heater disposed inside the inner cover develops heat that is kept with the inner cover made of the metallic plate. This enables the gas sensing element to be activated at an earlier stage.

A second aspect of the present invention provides a gas sensor comprising a gas sensing element operative to detect a concentration of a specified gas in measuring gases, a cylindrical housing internally supporting the gas sensing element in fixed place, and a cylindrical measuring gas side cover fixedly secured to the housing at a leading end thereof so as to cover a leading end of the gas sensing element. The gas sensor has a response, ranging from 150 ms to 200 ms, which is a parameter representing a speed of detecting the concentration of the specified gas with respect to variation in a specified gas concentration in the measuring gases. The measuring gas side cover has a multi-layer structure at least a part of which includes a mesh-like member formed with fine holes each having an opening surface area ranging from 0.1 mm² to 1 mm².

A third aspect of the present invention provides a gas sensor comprising a gas sensing element operative to detect a concentration of a specified gas in measuring gases, a cylindrical housing internally supporting the gas sensing element in fixed place, and a cylindrical measuring gas side cover fixedly secured to the housing at a leading end thereof so as to cover a leading end of the gas sensing element. The gas sensor has a response, ranging from 150 ms to 200 ms, which is a parameter representing a speed of detecting the concentration of the specified gas with respect to variation in a specified gas concentration in the measuring gases. The measuring gas side cover has a multi-layer structure including an outer cover and an inner cover, the outer cover including a cylindrical metallic plate body and a mesh-like cylindrical body formed with fine holes, each having an opening surface area ranging from 0.1 mm² to 1 mm², which is connected to one end of the metallic plate body.

A fourth aspect of the present invention provides a gas sensor comprising a gas sensing element operative to detect a concentration of a specified gas in measuring gases, a cylindrical housing internally supporting the gas sensing element in fixed place, and a cylindrical measuring gas side cover fixedly secured to the housing at a leading end thereof so as to cover a leading end of the gas sensing element. The gas sensor has a response, ranging from 150 ms to 200 ms, which is a parameter representing a speed of detecting the concentration of the specified gas with respect to variation in a specified gas concentration in the measuring gases. The measuring gas side cover has a multi-layer structure including an outer cover and an inner cover, the outer cover including a cylindrical metallic plate body formed with a gas ventilation bore providing fluid communication between inside and outside areas, and the inner cover including a mesh-like cylindrical body formed with fine holes each having an opening surface area ranging from 0.1 mm² to 1 mm².

A fifth aspect of the present invention provides a gas sensor comprising a gas sensing element operative to detect a concentration of a specified gas in measuring gases, a cylindrical housing internally supporting the gas sensing element in fixed place, and a cylindrical measuring gas side cover fixedly secured to the housing at a leading end thereof so as to cover a leading end of the gas sensing element. The gas sensor has a response, ranging from 150 ms to 200 ms, which is a parameter representing a speed of detecting the concentration of the specified gas with respect to variation in a specified gas concentration in the measuring gases. The measuring gas side cover has a multi-layer structure including an outer cover and an inner cover, the inner cover including a cylindrical metallic plate body formed with a gas ventilation bore providing fluid communication between inside and outside areas, and the outer cover including a mesh-like cylindrical body formed with fine holes each having an opening surface area ranging from 0.1 mm² to 1 mm².

### BRIEF DESCRIPTION OF THE DRAWINGS

FIG. 1 is a longitudinal cross sectional view showing an overall structure of a gas sensor of one embodiment according to the present invention.

FIG. 2A is an external view showing one example of a measuring gas side cover forming a part of the gas sensor shown in FIG. 1.

FIG. 2B is an enlarged view showing an exemplified structure of the measuring gas side cover shown in FIG. 2A.

FIG. 3 is an external view showing another example of the measuring gas side cover forming the part of the gas sensor shown in FIG. 1.

FIG. 4 is an external view showing another example of the measuring gas side cover forming the part of the gas sensor shown in FIG. 1.

FIG. 5 is an external view showing another example of the measuring gas side cover forming the part of the gas sensor shown in FIG. 1.

FIG. 6 is an external view showing a further example of the measuring gas side cover forming the part of the gas sensor shown in FIG. 1.

FIG. 7 is an external view showing a further example of the measuring gas side cover forming the part of the gas sensor shown in FIG. 1.

FIG. 8 is an external view showing a still further example of the measuring gas side cover forming the part of the gas sensor shown in FIG. 1.

FIG. 9 is a longitudinal cross sectional view showing an overall structure of a gas sensor of another embodiment according to the present invention.

FIG. 10 is a graph showing water adhesion rates of a gas sensor, implementing the present invention, and a gas sensor of the related art arising when suffered with the occurrence of water incursion.

FIG. 11 is a graph showing the relationship between a surface area of a fine hole and the number of fine holes formed in the measuring gas side cover and a response and water-incursion of the gas sensor.

### DETAILED DESCRIPTION OF PREFERRED EMBODIMENTS

Now, a gas sensor of one embodiment according to the present invention and a related method of manufacturing the gas sensor are described below in detail with reference to the accompanying drawings. However, the present invention is construed not to be limited to such an embodiment described below and technical concepts of the present invention may be implemented in combination with other known technologies or the other technology having functions equivalent to such known technologies.

In the following description, it is construed that a portion of the gas sensor adapted to be inserted to an exhaust pipe of an internal combustion engine of a motor vehicle is referred to as a “leading end ” or a “leading end portion” and an opposite side of the gas sensor exposed to an atmosphere is referred to as a “base end” or a “base end portion”.

Also, it will be appreciated that the gas sensor of the present embodiment according to the present invention may have a wide variety of applications to an oxygen sensor, an A/F sensor, a NOx sensor, etc.

### First Embodiment

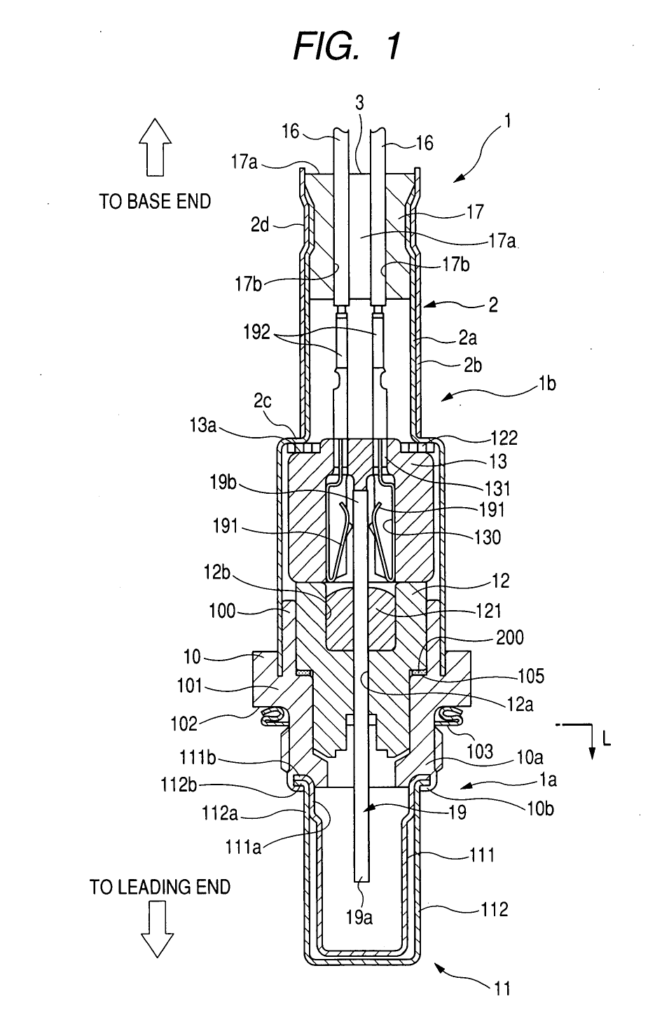

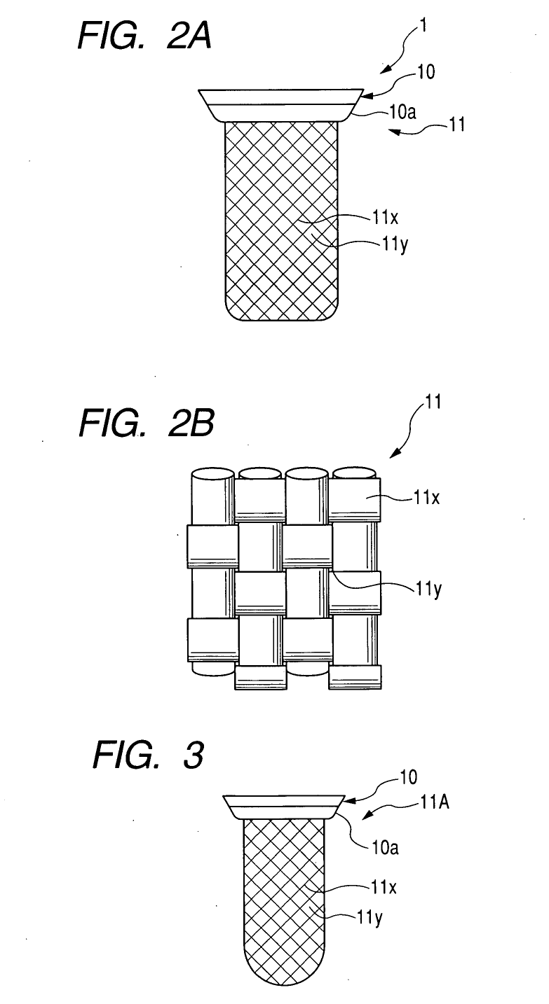

A gas sensor of one embodiment according to the present invention is described below in detail with reference to FIGS. 1 and 2.

FIG. 1 is a longitudinal cross sectional view showing an overall structure of the gas sensor of the present embodiment according to the invention. FIGS. 2A is an external view showing a measuring gas side cover for covering a gas sensing element of the gas sensor shown in FIG. 1. FIG. 2B is an enlarged view showing an exemplified lattice structure of the gas measuring side cover for the gas sensing element of the present embodiment.

As shown in FIG. 1, a gas sensor 1 of the present embodiment comprises a gas sensing element 19 for detecting a concentration of specified gas in measuring gases, a cylindrical housing 10 internally holding the gas sensing element 19, a cylindrical measuring gas side cover 11 fixedly secured to the cylindrical housing 10 at a leading end thereof so as to cover a leading end 19a of the gas sensing element 19, and a cylindrical atmospheric side cover 2 fixedly secured to the housing 10 at a base end thereof so as to cover a base end 19b of the gas sensing element 19.

Hereunder, these component parts with features thereof will be described below in detail. With the gas sensor 1 of the present embodiment, the measuring gas side cover 11 is formed in a mesh-like configuration by weaving wire components 11x. The wire components 11x have a clearance 11y equal to or less than 0.5 mm.

Further, the wire components 11x are made of stainless steel wires each with a diameter equal to or greater than 0.3φ.

With the gas sensor 1 formed in such a structure, the measuring gas side cover 11, composed of the wire components 11x woven into a mesh-like structure, provides the clearance 11y equal to or less than 0.5 mm between the wire components 11x. Thus, water drops can be prevented from penetrating into an inside of the measuring gas side cover 11 from the outside to cause a damage to occur on the gas sensing element 19. Accordingly, it becomes possible to provide a gas sensor with increased water-incursion resistance for preventing a gas sensing element from suffering water-incursion.

Further, the wire components 11x employ material such as stainless steel, providing heat resistant property. This allows the gas sensor 1 to be used under severely high temperature environments such as those environments exceeding a temperature equal to or higher than 1000° C. In addition, the use of the wire components 11x each with the diameter equal to or greater than 0.3φ makes it possible to suppress the measuring gas side cover 11 from deforming when subjected to impact shocks applied from the outside.

Hereunder, the gas sensor 1 will be described with reference to actual applications. With the gas sensor 1 of the present embodiment, in use, the cylindrical housing 10 is mounted on a wall surface of an exhaust pipe (not shown) extending from an automotive engine. Under such a mounting state, specified gas contained in exhaust gases (measuring gases) passing across the gas sensor 1 enters the inside of the measuring gas side cover 11 and is brought into contact with the leading end 19a of the gas sensing element 19. When this takes place, the gas sensing element 19 measures an air/fuel ratio of specified gas emitted from the automotive engine to provide an air/fuel ratio detection signal for use in controlling an air/fuel ratio of an air/fuel mixture of the automotive engine. In use of the gas sensor I for the exhaust pipe of the engine, the cylindrical housing 10 is mounted onto the exhaust pipe so as to allow an end face 102 of a radially extending trunk section 101, formed on a side wall of the cylindrical housing 10, to be brought into contact with an external wall of the exhaust pipe. Under such a mounted condition, the measuring gas side cover 11 extends into the inside of the exhaust pipe to be exposed to measuring gases passing therethrough to allow the gas sensing element 19 to detect a concentration of specified gas in measuring gases. Moreover, a gasket 103 rests on the end face 102 of the housing 10 to allow the end face 102 to be fixedly secured onto the wall surface of the exhaust pipe in a gastight sealing effect.

As shown in FIG. 1, the gas sensor 1 has a leading end region I a, extending downward from a lower end face of the gasket 103 at a boundary line L in FIG. 1, to be susceptible to heat of exhaust gases passing through the exhaust pipe during operation to measure the air/fuel ratio of specified gas in measuring gases. The gas sensor 1 also has a base end region 1b extending above the boundary line L to be susceptible to atmospheric environments. With such arrangement, the gas sensor 1 is warmed up due to heat of exhaust gases during operation such that the remoter from the boundary line L toward the base end of the gas sensor 1, the lower will be the temperature. In this respect, an upper section of the gas sensor 1 extending above the boundary line L in FIG. 1 is referred to as the base end region 1b of the gas sensor 1 and a lower section is referred to as the leading end region 1a.

The measuring gas side cover 11 is fixedly mounted to an end face of a leading end portion 10a of the cylindrical housing 10. In addition, the measuring gas side cover 11 internally accommodates therein the leading end of the gas sensing element 19.

In particular, the measuring gas side cover 11 includes an inner cover 111, having a cylindrical base portion 11a formed with a radially outward annular flange 11b, and an outer cover 112 having a cylindrical base portion 112a, fitted to an outer periphery of the cylindrical base portion 111a of the inner cover 111, and a radially outward annular flange 112b overlapping with the annular flange 111b of the inner cover 111. The annular flanges 11b and 112b of the inner cover 111 and the outer cover 112 are fixedly supported with the leading end portion 10a of the cylindrical housing by a caulked end 10b of the cylindrical housing 10 such that the measuring gas side cover 11 extends in coaxial relation with the gas sensing element 19.

The gas sensing element 19 is fixedly mounted on the housing 10 by means of an element-side insulating porcelain holder 12 having an element inserting bore 12a through which the gas sensing element 19 longitudinally extends to be held in a fixed place. A metallic packing element 200 rests on a tapered annular shoulder 105 formed in the housing 10 to be sandwiched between the element-side insulating holder 12 and the housing 10. This provides a gastight sealing effect between the element-side insulating holder 12 and the housing 10, thereby preventing fluid communication between the leading end region I a and the base end region 1b of the gas sensor 1.

The element-side insulating holder 12 has a cylindrical cavity 12b that is filled with airtight sealant 121. Airtight sealant 121 provides a gastight sealing effect between the gas sensing element 19 and the element-side insulating holder 12 to prevent measuring gases from leaking through a clearance between the gas sensing element 19 and the element inserting bore 12a of the element-side insulating holder 12 to an upper area of the element-side insulating holder 12.

An atmospheric side porcelain insulator 13 is placed on the element-side insulating holder 12 in contact therewith. The atmospheric side porcelain insulator 13 has an axially extending cavity portion 130, which accommodates therein the base end portion 19b of the gas sensing element 19, and a plurality of connection holes 131 formed in an upper wall of the atmospheric side porcelain insulator 13 to provide connection between the cavity portion 130 and an end face of the atmospheric side porcelain insulator 13.

A cone-shaped disc spring 122 is disposed between an annular shoulder 2c of the cylindrical atmospheric side cover 2 and an annular shoulder 13a formed on the upper wall of the atmospheric side porcelain insulator 13 to provide a restoring force for axially pressing the atmospheric side porcelain insulator 13 toward the leading end region 1a of the gas sensor 1, that is, in a direction parallel to a central axis of the gas sensor 1. That is, the cone-shaped disc spring 122 allows the atmospheric side porcelain insulator 13 to press the element-side insulating holder 12 against the tapered annular shoulder 105 of the housing 10, thereby compressing the packing element 200 to provide a gastight sealing effect.

The axially extending cavity portion 130 of the atmospheric side porcelain insulator 13 accommodates therein a plurality of spring terminals 191, 191 held in electrical contact with electrode terminals (not shown) formed on the base end portion 19b of the gas sensing element 19 for supplying electric power thereto and extracting a detection output from the gas sensing element 19 to the outside. To this end, the spring terminals 191 are electrically connected through connecting members 192 to lead wires 16.

The lead wires 16 are taken out of the gas sensor 1 for connection to an externally located measuring device and a power supply or the like.

The atmospheric side cover 2 takes a double-layer structure including an inner cover 2a and an outer cover 2b. The inner cover 2a, substantially cylindrical in cross section and made of stainless steel (SUS304), is directly fixed to a peripheral wall of a base end portion 100 of the housing 10 by welding. The outer cover 2b, substantially cylindrical in cross section and made of stainless steel (SUS304), is fitted onto an outer circumference of a base end portion of the inner cover 2a and fixed thereto by caulking made at a caulked portion 2d.

The inner cover 2a has a base portion that accommodates therein a sealing member 17 which is fixedly retained with the caulked portion 2d of the atmospheric side cover 2. The sealing member 17 includes a rubber bush made of fluorine-contained rubber and has a columnar shape in cross section. The sealing member 17 has a central area formed with an axially extending atmospheric introduction bore 17a for introducing atmospheric air to an axially central area inside the atmospheric side cover 2. A plurality of lead wire insertion holes 17b, 17b is formed in the sealing member 17 at plural positions around the atmospheric introduction bore 17a.

The sealing member 17 has a base end face 17a that carries thereon a ventilation filter 3. The ventilation filter 3 is made of porous material such as, for instance, polytetrafluoroethylene (PTEF) and has high air ventilating capability that can permeates atmospheric air.

Meanwhile, with the gas sensor I of the present embodiment, the measuring gas side cover 11 takes a double-layer structure including the inner cover 111 and the outer cover 112. The outer cover 112 and/or the inner cover 111 are formed in mesh-like configurations by weaving the wire components 11x formed with a clearance 11y equal to or less than 0.5 mm. In addition, the wire components 11x are made of stainless steel wires each with a diameter equal to or greater than 0.3φ.

With the gas sensor 1 of the present embodiment formed in such a structure, weaving the wire components 11x allows the outer cover 112 and/or the inner cover 111 to be formed in the mesh-like configurations so as to permit the clearance between the adjacent wire components 11x to lie in a value equal to or less than 0.5 mm. This allows the measuring gas side cover 11 to have increased water-incursion resistance to prevent water droplets from penetrating to the inside of the inner cover 111. Accordingly, it becomes possible to provide a gas sensor that is less susceptible to water-incursion.

Further, the wire components 11x are made of material such as stainless steel. This enables the gas sensor 1 to be used under severely high temperature environments such as those exceeding a temperature equal to or higher than 1000° C. In addition, the use of the wire components 11x with the diameter equal to or greater than 0.3φ enables the suppression of the measuring gas side cover 11 from deforming even when subjected to impact shocks applied from the outside.

Furthermore, the measuring gas side cover 11 may take appropriately designed structure to have any suitable shape in cross section.

### Second Embodiment

FIGS. 3 to 5 are external views showing measuring gas side covers for use in gas sensors of other embodiments according the present invention.

FIG. 3 shows one example of a measuring gas side cover 11A formed in a saclike structure. In fabricating the measuring gas side cover 11A with such a saclike structure shown in FIG. 3, the wire components 11x, made of stainless steel, are woven into a mesh-like sheet. The mesh-like sheet is then pressed against a dome-shaped die (not shown) and rounded into a final saclike shape as shown in FIG. 3 with the mesh-like sheet being maintained in a uniform mesh pattern.

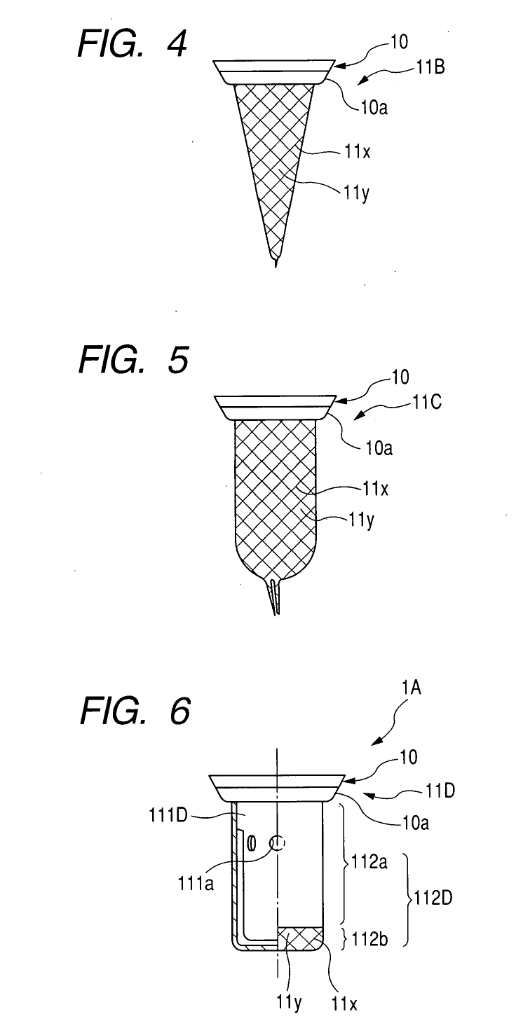

Further, FIG. 4 shows another example of a measuring gas side cover 11B formed in a cone-shaped configuration. In fabricating the measuring gas side cover 11B with such a cone-shaped structure shown in FIG. 4, the mesh-like sheet, composed of the woven wire components 11x made of stainless steel, is wound on a cone-shaped die (not shown), making it easy to fabricate the measuring gas side cover 11B.

Furthermore, FIG. 5 shows still another example of a measuring gas side cover 11C composed of the stainless mesh sheet. The stainless mesh sheet is formed in a cylindrical shape with a leading end 11s being shackled and closed. In fabricating the measuring gas side cover 11C, the stainless mesh sheet, composed of the woven wire components 11x, is pressed against the dome-shaped die and rounded into a cylindrical shape as shown in FIG. 5, after which the leading end 11s is shackled and closed, making it easy to fabricate the measuring gas side cover 11C.

### Third Embodiment

FIGS. 6 to 8 are external views showing measuring gas side covers 11D, 11E, 11F for use in gas sensors of other embodiments according the present invention.

In FIGS. 6 to 8, right areas beyond a centerline show the measuring gas side covers in external appearances and left areas beyond the centerline represent internal structures of the measuring gas side covers.

With a gas sensor 1A shown in FIG. 6, the measuring gas side cover 11D includes an outer cover 112D. The outer cover 112D includes a cylindrical metallic plate body 112a, having a base end fixedly secured to the leading end portion 10a of the housing 10, and a mesh-like cylindrical member 112b, made of the woven stainless steel wire components 11x, which is fixedly secured to a leading end of the cylindrical metallic plate body 112a.

The measuring gas side cover 111D further includes an inner cover 111D disposed inside the outer cover 112D. The inner cover 111D has a plurality of gas ventilation bores 111a through which measuring gases pass into an inside area of the measuring gas side cover 11D. The cylindrical metallic plate body 112a is so shaped as to cover the gas ventilation bores 111a of the inner cover 111D in a radial direction. This allow measuring gases to enter through the mesh-like cylindrical member 112b of the outer cover 112D and pass through the gas ventilation bores 111a into the inside area of the inner cover 111D.

With such a measuring gas side cover 111D, the gas ventilation bores 111a, formed in the inner cover 111D, can be protected with the cylindrical metallic plate body 112a forming the outer cover 112a. This allows the measuring gas side cover 11D to have increased water-incursion resistance.

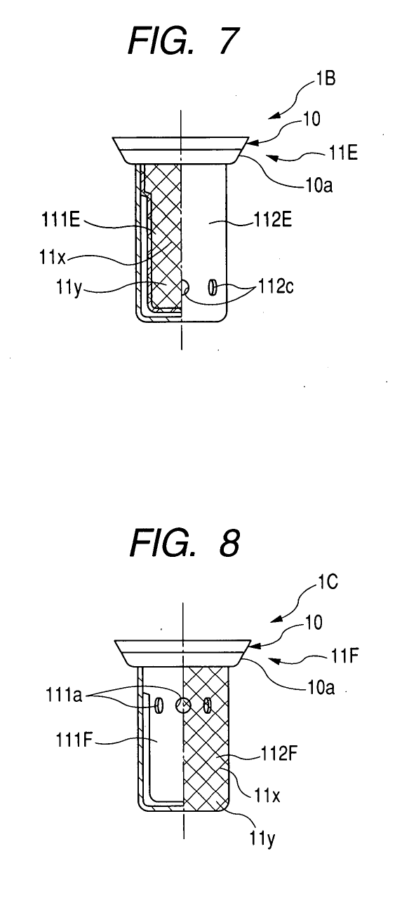

With a gas sensor 1B shown in FIG. 7, a measuring gas side cover 11E takes the form of a multi-layer structure formed in a cylindrical configuration. The measuring gas side cover 11E includes two kinds of an inner cover 111E and an outer cover 112E.

The inner cover 111E is composed of a mesh-like sheet composed of the woven wire components 11x.

The outer cover 112E includes a cylindrical metallic plate body, made by press forming a metallic plate into a cylindrical shape, which is formed with a plurality of gas ventilation bores 112c.

The inner cover 111E and the outer cover 112E are fitted to each other at both base ends thereof and fixedly secured to the leading end portion 10a of the housing 10.

With the gas sensor 1B of such a structure shown in FIG. 7, measuring gases pass through the plurality of gas ventilation bores 112c formed in the outer cover 112E to an inside area of the outer cover 112E. Then, measuring gases, entered an internal space between the inner cover 11b and the outer cover 112E, pass through the clearances 11y of the woven wire components 11x, forming the inner cover 111E, into an inside area of the inner cover 111E to reach the leading end of the gas sensing element (not shown). Thus, the gas sensor 11B has improved response in operation.

With a gas sensor 1C shown in FIG. 8, a measuring gas side cover 11F takes the form of a multi-layer structure formed in a cylindrical configuration. The measuring gas side cover 11F includes two kinds of an inner cover 111F and an outer cover 112F.

Further, the inner cover 111F internally accommodates therein the gas sensing element (not shown) and a heater (not shown) for raising a temperature of the gas sensing element.

The inner cover 111F includes a cylindrical metallic plate body, made by press forming a metallic sheet plate, and has a plurality of gas ventilation bores 111a.

The outer cover 1112 is made of a mesh-like sheet formed by weaving the wire components 11x.

The inner cover 111F and the outer cover 112F are fitted to each other at both base ends thereof and fixedly secured to the leading end portion 10a of the housing 10.

With the measuring gas side cover 11F of such a gas sensor 1C, a heat developed by the heater provided inside the inner cover 111F is kept with the inner cover 111F made of the metallic plate. This allows the gas sensing element to be activated on an earlier stage after startup of the engine.

### Fourth Embodiment

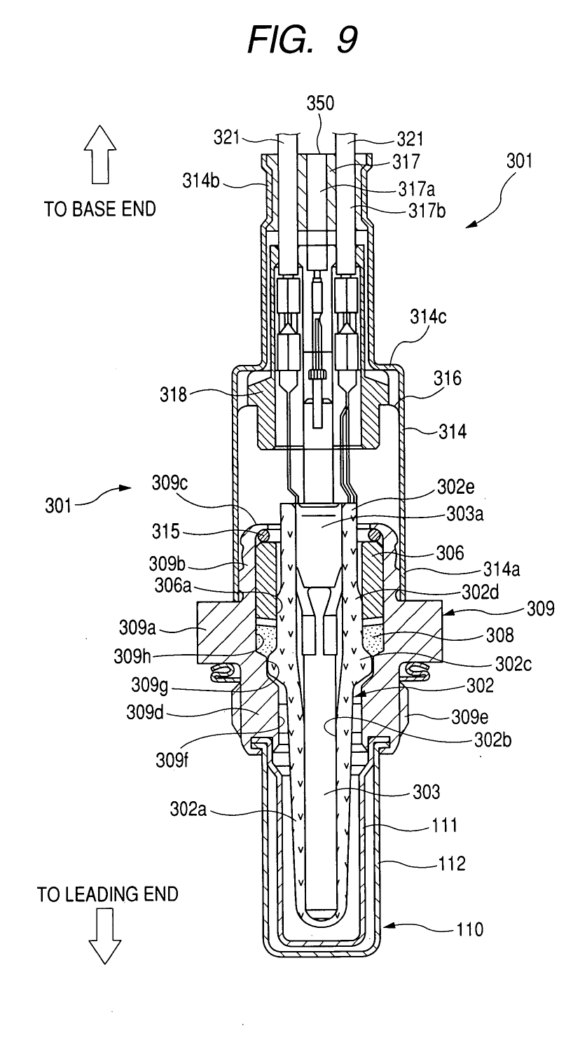

FIG. 9 is a longitudinal cross sectional view showing an overall structure of a gas sensor of a fourth embodiment according to the present invention.

As shown in FIG. 9, the gas sensor 301 of the present embodiment comprises a hollow gas sensing element 302 with a leading end 302a closed and internally formed with an axial bore 302b, and a heating element 303 embedded in the axial bore 302b of the gas sensing element 302 and composed of a bar-like ceramic heater.

The gas sensing element 302 is made of a solid electrolyte having an oxygen ion conductivity.

The gas sensing element 302 has a radially extending annular protrusion 302c formed at a base end of the leading portion 302a to have a larger diameter than that of the leading portion 302a. An intermediate hollow portion 302d axially extends from the annular protrusion 302c in opposition to the leading end 302a. The gas sensing element 302 has a hollow base end portion 302e with which a base end portion 303a of the heater 303 is rigidly supported.

The gas sensor 301 further includes an element insulating holder 306, made of porcelain insulating material such as ceramic, which has a hollow space 306a in which the intermediate hollow portion 302d of the gas sensing element 302 is rigidly supported. The element insulating holder 306 is accommodated in and rigidly supported with a metallic housing 309.

The metallic housing 309 includes a main housing body 309a, acting as a gas sensing element accommodating body, which has a base end portion 309b having a terminal end formed with a radially inward annular flange 309c and a leading end portion 309d having an outer periphery formed with a threaded portion 309e adapted to be screwed onto a mounting area of an exhaust pipe of an internal combustion engine.

The housing 309 has a small diameter bore 309f formed inside the leading end portion 309d, an intermediate bore 309g formed inside the main housing body 309a for retaining the annular protrusion 302c of the gas sensing element 302, and a large diameter bore 309h formed inside the main housing body 309a and the base end portion 309b.

A gastight sealant 308, made of ceramic powder such as talc, is filled in an annular space between an outer periphery of the intermediate hollow portion 302d and the large diameter bore 309g of the metallic housing 309 to provide a gastight sealing effect. The element insulating holder 306 is fitted to the large diameter bore 309g of the metallic housing 309 so as to compact the gastight sealant 308. In addition, the gas sensor 1 further includes an atmospheric side cover 314 having a leading end fixedly secured to the base end portion 309b of the metallic housing 309 by welding, and the measuring gas side cover 11 fixedly secured to a terminal end of the leading end portion 309d.

Further, a pressure ring 315 is held in pressured contact with the annular flange 309c of the metallic housing 309 to press the element insulating holder 306 against the gastight sealant 308. Thus, the element insulating holder 306 and the gastight sealant 308 are fixed to the metallic housing 309 at a base end thereof.

The atmospheric side cover 314 has a large diameter leading end 314a fitted to and fixed to the base end portion 309b of the metallic housing 309. The atmospheric side cover 314 also has a small diameter base end portion 314b with an open end that is caulked to fixedly hold a sealing member 317 made of resilient material such as rubber or the like for providing a sealing effect. The atmospheric side cover 314 accommodates therein an insulator 318 at a position in close proximity to an annular shoulder portion 314c between the leading end portion 314a and the base end portion 314b. The insulator 318 is held with the atmospheric side cover 314 by means of a pressure spring 316 disposed between the atmospheric side cover 314 and the insulator 318.

Further, the sealing member 317 has a ventilation bore 317a and a plurality of lead insertion bores 317b, formed in areas around the ventilation bore 317a, through which lead wires 321 extend.

Meanwhile, with the gas sensor 301 of the present embodiment, the measuring gas side cover 11 takes the same double-layer structure as that of the gas sensor 1 of the first embodiment shown in FIG. 1 and includes the inner cover 111 and the outer cover 112. In addition, the inner cover 111 and/or the outer cover 112 are formed in the mesh-like configuration by weaving the wire components 11x, mentioned above, which have the clearance 11y equal to or less than 0.5 mm. In addition, the wire components 11x are made of stainless steel and each of the wire components 11x has a diameter equal to or greater than 0.3φ.

With the gas sensor 301 formed in such a structure, the measuring gas side cover 11 is formed in the mesh-like configuration by weaving the sire components 11x so as to provide the clearance 11y equal to or less than 0.5 mm. Thus, the measuring gas side cover 11, formed in the mesh-like configuration with such a clearance, effectively prevents water droplets from entering the inside of the measuring gas side cover 11. This makes it possible to provide a gas sensor that can prevent the gas sensing element from suffering water-incursion.

Further, the wire components 11x are made of material such as stainless steel, providing heat resistant property. This enables the gas sensor 301 to be used under severe environments such as those exceeding a temperature equal to or higher than 1000° C. In addition, the use of the wire components 11x with the diameter equal to or greater than 0.3φ makes it possible to suppress the measuring gas side cover 11 from deforming even when subjected to impact shocks applied from the outside.

### EXAMPLE

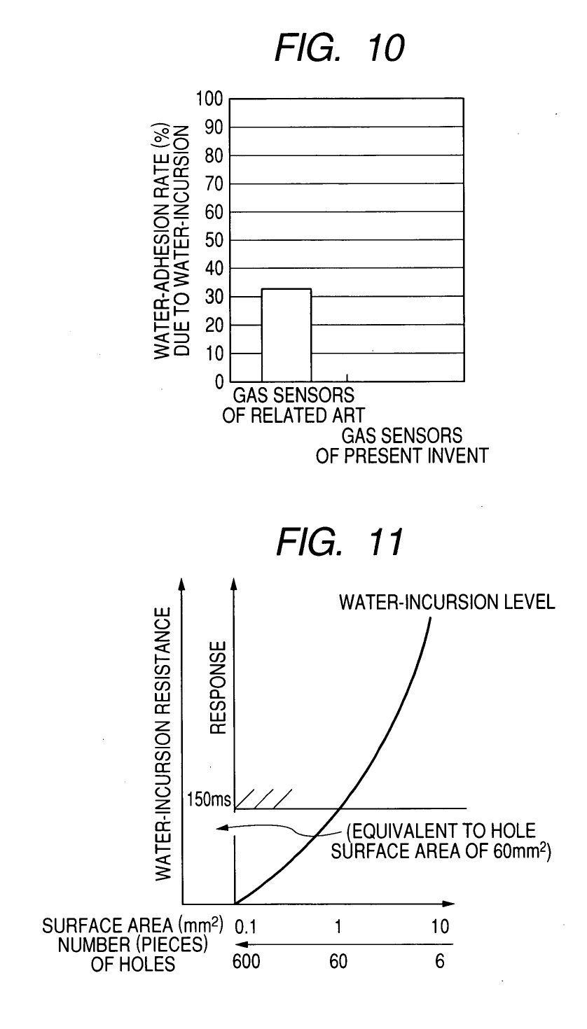

FIG. 10 is a graph showing evaluated comparison results between the gas sensor of the present embodiment and the gas sensor of the related art.

For comparison purposes, 30 samples of the gas sensor of the related art were manufactured each with the same dimension as that of the gas sensor of the present invention and had a measuring gas side cover formed with six gas ventilation bores each having a diameter of φ3 mm. Meanwhile, 30 samples of the gas sensor of the present invention were manufactured each having a measuring gas side cover formed in a mesh-like structure with dimensions of relevant parts mentioned above. Upon using these two types of the gas sensors, tests were conducted to obtain evaluations described below.

In particular, first, powder was coated on the gas sensing elements. Then, the gas sensing elements were mounted on an exhaust pipe of an internal combustion engine and the gas sensing elements were heated to a temperature of 700° C. using a heater. Subsequently, water was poured into an inside of the exhaust pipe. Next, a blower was driven to blow off water droplets onto the gas sensing elements for a time period of three minutes. Then, the gas sensors were collected to confirm whether or not cracking occurred on the gas sensing elements. Tests were conducted on 30 samples of each of the gas sensors of the related art and the gas sensors of the present embodiment in the same sequence mentioned above.

As a result of tests, among the 30 pieces of the examples of the related art, 10 samples of the gas sensor of the related art encountered with cracking occurring in the gas sensing elements with a cracking incidence rate of approximately 30% as will be apparent from the graph of FIG. 10. On the contrary, no cracking was found on the samples of the gas sensor of the present embodiment.

While the specific embodiments of the present invention have been described in detail, the present invention is not limited to the particularly illustrated structures of the gas sensors of the various embodiment set forth above provided that the measuring gas side covers achieve the task of the present invention. It will be appreciated by those skilled in the art that various modifications and alternatives to those details could be developed in light of the overall teachings of the disclosure. For instance, the wire components are not limited to stainless steel and may be made of other heat resistant material such as Inconel or the like. In addition, measuring gases to be detected are not limited to oxygen and may include other gases such as NOx, CO and HC or the like. Moreover, the gas sensing element may include any one of a stack type, a cup type, etc. Thus, the particular arrangements disclosed are meant to be illustrative only and not limited to the scope of the present invention, which is to be given the full breadth of the following claims and all equivalents thereof.

What is claimed is:

1. A gas sensor comprising:
a gas sensing element operative to detect a concentration of a specified gas in measuring gases;
a cylindrical housing internally supporting the gas sensing element in fixed place; and
a cylindrical measuring gas side cover fixedly secured to the housing at a leading end thereof so as to cover a leading end of the gas sensing element;
wherein the gas sensor has a response, ranging from 150 ms to 200 ms, which is a parameter representing a speed of detecting the concentration of the specified gas with respect to variation in a specified gas concentration in the measuring gases; and
wherein the measuring gas side cover has fine holes each with an opening surface area ranging from 0.1 mm² to 1 mm².

2. The gas sensor according to claim 1, wherein:
the measuring gas side cover is made of a mesh-like member composed of wire components woven with a clearance equal to or less than 1 mm; and
the wire components are made of stainless steel wires each having a diameter equal to or greater than 0.5φ.

3. The gas sensor according to claim 1, wherein:
the measuring gas side cover has a saclike configuration.

4. The gas sensor according to claim 1, wherein:
the measuring gas side cover has a cone-shaped configuration.

5. The gas sensor according to claim 1, wherein:
the measuring gas side cover has a cylindrical configuration with a leading end being shackled and closed.

6. The gas sensor according to claim 1, wherein:
the measuring gas side cover is formed in a cylindrical shape and includes a cylindrical metallic plate body and a mesh-like cylindrical body, composed of woven wire components, which is connected to one end of the metallic plate body.

7. The gas sensor according to claim 7, wherein:
the measuring gas side cover is formed in a cylindrical shape and includes an inner cover formed in a cylindrical shape and disposed in an inside area;
wherein the inner cover has a gas ventilation bore providing fluid communication between inside and outside areas; and
wherein the metallic plate, body acts as an outer cover that is radially spaced from the inner cover so as to cover the gas ventilation bore.

8. The gas sensor according to claim 1, wherein:
the measuring gas side cover includes a multi-layer structure formed in a cylindrical shape having two kinds of an inner cover and an outer cover;
wherein the inner cover includes a mesh-like member formed by weaving wire components; and
wherein the outer cover is made of a metallic plate and has a gas ventilation bore.

9. The gas sensor according to claim 1, wherein:
the measuring gas side cover includes a multi-layer structure formed in a cylindrical shape having two kinds of an inner cover and an outer cover;
wherein the inner cover is made of a metallic plate and has a gas ventilation bore; and
wherein the outer cover includes a mesh-like member formed by weaving wire components.

10. A gas sensor comprising:
a gas sensing element operative to detect a concentration of a specified gas in measuring gases;
a cylindrical housing internally supporting the gas sensing element in fixed place; and
a cylindrical measuring gas side cover fixedly secured to the housing at a leading end thereof so as to cover a leading end of the gas sensing element;
wherein the gas sensor has a response, ranging from 150 ms to 200 ms, which is a parameter representing a speed of detecting the concentration of the specified gas with respect to variation in a specified gas concentration in the measuring gases; and
wherein the measuring gas side cover has a multi-layer structure at least a part of which includes a mesh-like member formed with fine holes each having an opening surface area ranging from 0.1 mm² to 1 mm².

11. The gas sensor according to claim 10, wherein:
the mesh-like member is composed of wire components woven with a clearance equal to or less than 1 mm; and
the wire components are made of stainless steel wires each having a diameter equal to or greater than 0.5φ.

12. The gas sensor according to claim 10, wherein:
the multi-layer structure has a saclike configuration. equal to or greater than 0.5φ.

17. The gas sensor according to claim 16, wherein:
the inner cover has a gas ventilation bore providing fluid communication between inside and outside areas; and
wherein the outer cover is radially spaced from the inner cover so as to cover the gas ventilation bore.

18. A gas sensor comprising:
a gas sensing element operative to detect a concentration of a specified gas in measuring gases;
a cylindrical housing internally supporting the gas sensing element in fixed place; and
a cylindrical measuring gas side cover fixedly secured to the housing at a leading end thereof so as to cover a leading end of the gas sensing element;
wherein the gas sensor has a response, ranging from 150 ms to 200 ms, which is a parameter representing a speed of detecting the concentration of the specified gas with respect to variation in a specified gas concentration in the measuring gases; and
wherein the measuring gas side cover has a multi-layer structure including an outer cover and an inner cover, the outer cover including a cylindrical metallic plate body formed with a gas ventilation bore providing fluid communication between inside and outside areas, and the inner cover including a mesh-like cylindrical body formed with fine holes each having an opening surface area ranging from 0.1 mm² to 1 mm².

19. The gas sensor according to claim 18, wherein:
the mesh-like cylindrical body is composed of wire components woven with a clearance equal to or less than 1 mm; and the wire components are made of stainless steel wires each having a diameter equal to or greater than 0.5φ.

20. A gas sensor comprising:
a gas sensing element operative to detect a concentration of a specified gas in measuring gases;
a cylindrical housing internally supporting the gas sensing element in fixed place; and
a cylindrical measuring gas side cover fixedly secured to the housing at a leading end thereof so as to cover a leading end of the gas sensing element;
wherein the gas sensor has a response, ranging from 150 ms to 200 ms, which is a parameter representing a speed of detecting the concentration of the specified gas with respect to variation in a specified gas concentration in the measuring gases; and
wherein the measuring gas side cover has a multi-layer structure including an outer cover and an inner cover, the inner cover including a cylindrical metallic plate body formed with a gas ventilation bore providing fluid communication between inside and outside areas, and the outer cover including a mesh-like cylindrical body formed with fine holes each having an opening surface area ranging from 0.1 mm² to 1 mm².

13. The gas sensor according to claim 10, wherein:
the multi-layer structure has a cone-shaped configuration.

14. The gas sensor according to claim 10, wherein:
the multi-layer structure has a cylindrical configuration with a leading end being shackled and closed.

15. A gas sensor comprising:
a gas sensing element operative to detect a concentration of a specified gas in measuring gases;
a cylindrical housing internally supporting the gas sensing element in fixed place; and
a cylindrical measuring gas side cover fixedly secured to the housing at a leading end thereof so as to cover a leading end of the gas sensing element;
wherein the gas sensor has a response, ranging from 150 ms to 200 ms, which is a parameter representing a speed of detecting the concentration of the specified gas with respect to variation in a specified gas concentration in the measuring gases; and
wherein the measuring gas side cover has a multi-layer structure including an outer cover and an inner cover, the outer cover including a cylindrical metallic plate body and a mesh-like cylindrical body formed with fine holes, each having an opening surface area ranging from 0.1 mm² to 1 mm², which is connected to one end of the metallic plate body.

16. The gas sensor according to claim 15, wherein:
the mesh-like cylindrical body is composed of wire components woven with a clearance equal to or less than 1 mm; and
the wire components are made of stainless steel wires each having a diameter
